(19) United States
(12) Patent Application Publication
(10) Pub. No.: US 2025/0101239 A1
(43) Pub. Date: Mar. 27, 2025

(54) Method of Making Paint Having Heat-Dissipating Graphene

(71) Applicant: Chih-Ming Lee, Taichung City (TW)

(72) Inventor: Chih-Ming Lee, Taichung City (TW)

(21) Appl. No.: 18/370,886

(22) Filed: Sep. 21, 2023

(51) Int. Cl. C09D 7/80; C09D 1/00; C12N 1/20; C12P 3/00

(52) U.S. Cl. CPC C09D 7/80; C09D 1/00; C12N 1/205; C12P 3/00

(57) ABSTRACT

A method of making paint contains steps of a first-time/second fermentation. Graphene mother bacteria are added to warm water and are placed in a temperature-controlled room for two days to cultivate bacteria. Then, the steps of the first-time/second fermentation include: i) pouring the graphene mother bacteria, edible powders, and powdered sugars into a container, then adding the warm waters into the container; ii) crushing the graphene fermentation clump evenly by using a crush tool to produce crushed graphene fermentation pieces, spreading the crushed graphene fermentation pieces on a stainless steel platform and placing the crushed graphene fermentation pieces in the temperature-controlled room to dry for three to four days; iii) putting the enzyme product into a mixer so as to stir and refine the enzyme product into enzyme powders. After finishing the first-time fermentation, the steps i) to iii) are repeated to complete the steps of the second-time fermentation.

### TECHNICAL FIELD

The present disclosure relates to a method of making paint which is non-toxic, non-conductive and heat-dissipating, and it creates a protective layer for rapid heat dissipation, when applying the oil paint on an exterior wall.

### BACKGROUND

Conventionally, there are two types of paints: “water-based” and “oil-based”. The difference lies in the substances used to dilute the paint when applying. The water-based paint uses “water” as a thinner, and the oil-based paint uses “organic solvents” (toluene, rosin water, etc.) as thinners. Taking health into consideration, the paint manufacturing process does not add formaldehyde or heavy metal harmful substances, and the organic volatile substances are low, so as to protect the health of consumers at home.

The present invention has arisen to mitigate and/or obviate the afore-described disadvantages.

### SUMMARY

A primary aspect of the present invention is to provide a method of making paint which is non-toxic, non-conductive and heat-dissipating, and it creates a protective layer for rapid heat dissipation, when applying the oil paint on an exterior wall.

To obtain above-mentioned aspect, a method of making paint provided by the present invention contains: steps of a first-time/second fermentation.

Graphene mother bacteria are added to warm water and are placed in a temperature-controlled room for two days to cultivate bacteria. Then, the steps of the first-time/second fermentation include: i) pouring the graphene mother bacteria, edible powders, and powdered sugars into a container, then adding the warm waters into the container; ii) crushing the graphene fermentation clump evenly by using a crush tool to produce crushed graphene fermentation pieces, spreading the crushed graphene fermentation pieces on a stainless steel platform and placing the crushed graphene fermentation pieces in the temperature-controlled room to dry for three to four days; iii) putting the enzyme product into a mixer so as to stir and refine the enzyme product into enzyme powders. After finishing the first-time fermentation, the steps i) to iii) are repeated to complete the steps of the second-time fermentation.

Preferably, banana water, graphene, Shungite, and fermented boron nitride powder in 5 to 10 grams are added into oil paint coating to produce oil paint.

Preferably, clean water, graphene, Shungite, and fermented boron nitride powder in 5 to 10 grams are added into water paint coating to produce water paint

Preferably, the graphene mother bacteria is placed in the temperature-controlled room for two days to the cultivate bacteria.

Preferably, the graphene baking powders is placed in the temperature-controlled room for three days to ferment.

Preferably, the crushed graphene fermentation pieces are placed in the temperature-controlled room to dry for three to four days.

Preferably, the temperature-controlled room is at a temperature of 38° C. to 40° C.

Preferably, in the step i), a mixing proportion of the graphene mother bacteria, the edible powders, and the powdered sugars in the warm waters is the graphene mother bacteria in 150 grams, the edible powders in 3 kgs, the powdered sugars in 100 grams, and the warm water in 3000 cc.

Preferably, the platform is made of stainless steel.

Preferably, the container is a plastic blanket or a glass blanket.

Other objects and features will be in part apparent and in part pointed out hereinafter.

### BRIEF DESCRIPTION OF THE DRAWINGS

FIG. 1 is a flow chart of a method of making graphene baking powders according to a preferred embodiment of the present invention.

FIG. 2 is a flow chart of a mixing proportion of the graphene baking powders according to the preferred embodiment of the present invention.

FIG. 3 is a flow chart of a method of making a paint having heat-dissipating graphene according to the preferred embodiment of the present invention.

FIG. 4 is a block diagram showing the application of the paint according to another preferred embodiment of the present invention.

FIG. 5 is another block diagram showing the application of the paint according to another preferred embodiment of the present invention.

### DETAILED DESCRIPTION

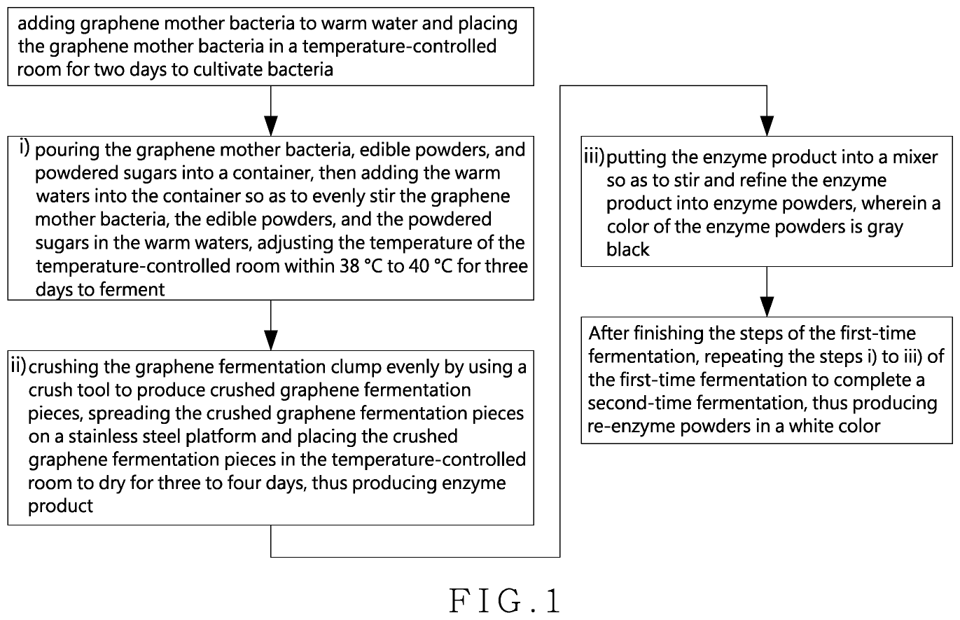

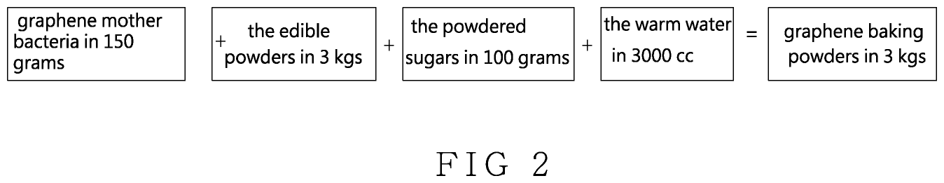

FIG. 1 is a flow chart of a method of making paint having heat-dissipating graphene according to a preferred embodiment of the present invention. The method of making the paint comprises steps of a first-time/second fermentation:
adding graphene mother bacteria to warm water and placing the graphene mother bacteria in a temperature-controlled room for two days to cultivate bacteria;
i) pouring the graphene mother bacteria, edible powders, and powdered sugars into a container which is a plastic blanket or a glass blanket, then adding the warm waters into the container so as to evenly stir the graphene mother bacteria, the edible powders, and the powdered sugars in the warm waters (as shown in FIG. 2), wherein a mixing proportion of the graphene mother bacteria, the edible powders, and the powdered sugars in the warm waters is the graphene mother bacteria in 150 grams, the edible powders in 3 kgs, the powdered sugars in 100 grams, and the warm water in 3000 cc, thus producing graphene baking powders; thereafter the graphene baking powders is placed in the temperature-controlled room at a temperature of 38° C. to 40° C. for three days to ferment, thus producing graphene fermentation clump.
ii) crushing the graphene fermentation clump evenly by using a crush tool to produce crushed graphene fermentation pieces, spreading the crushed graphene fermentation pieces on a stainless steel platform and placing the crushed graphene fermentation pieces in the temperature-controlled room to dry for three to four days, thus producing enzyme product;
iii) putting the enzyme product into a mixer so as to stir and refine the enzyme product into enzyme powders, wherein a color of the enzyme powders is gray black.

After finishing the steps of the first-time fermentation, repeating the steps i) to iii) of the first-time fermentation to complete a second-time fermentation, thus producing re-enzyme powders in a white color.

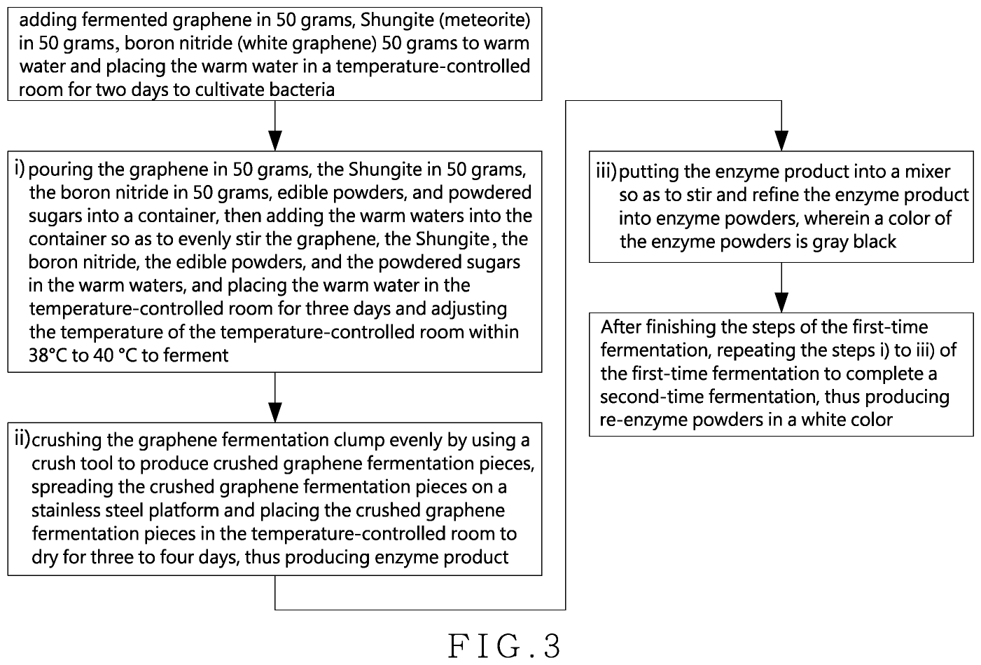

FIG. 3 is a flow chart of a method of making a paint having heat-dissipating graphene according to the preferred embodiment of the present invention.

The method of making the paint comprises steps of:
adding fermented graphene in 50 grams, Shungite (meteorite) in 50 grams, boron nitride (white graphene) 50 grams to warm water and placing the warm water in a temperature-controlled room for two days to cultivate bacteria;
i) pouring the graphene in 50 grams, the Shungite in 50 grams, the boron nitride in 50 grams, edible powders, and powdered sugars into a container, then adding the warm waters into the container so as to evenly stir the graphene, the Shungite, the boron nitride, the edible powders, and the powdered sugars in the warm waters, and placing the warm water in the temperature-controlled room for three days and adjusting the temperature of the temperature-controlled room within 38° C. to 40° C. to ferment;
ii) crushing the graphene fermentation clump evenly by using a crush tool to produce crushed graphene fermentation pieces, spreading the crushed graphene fermentation pieces on a stainless steel platform and placing the crushed graphene fermentation pieces in the temperature-controlled room to dry for three to four days, thus producing enzyme product; and
iii) putting the enzyme product into a mixer so as to stir and refine the enzyme product into enzyme powders, wherein a color of the enzyme powders is gray black.

After finishing the steps of the first-time fermentation, repeating the steps i) to iii) of the first-time fermentation to complete a second-time fermentation, thus producing re-enzyme powders in a white color.

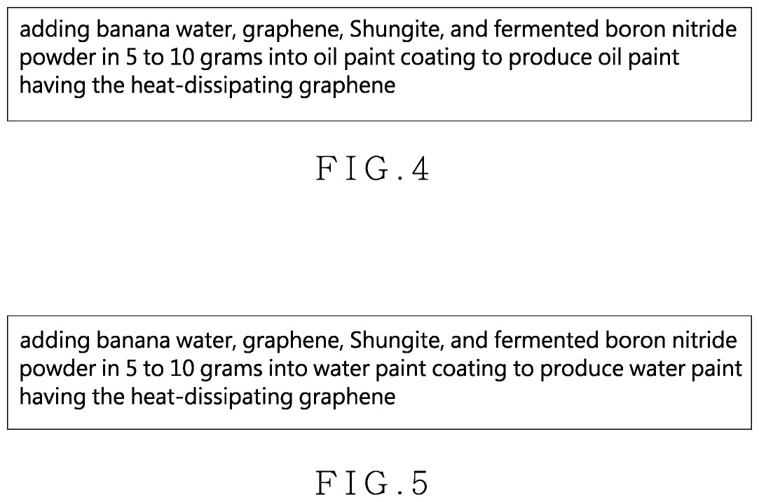

FIG. 4 is a block diagram showing the application of the paint according to another preferred embodiment of the present invention. In this application, banana water, graphene, Shungite, and fermented boron nitride powder in 5 to 10 grams are added into oil paint coating to produce oil paint having the heat-dissipating graphene. Preferably, the oil paint is non-toxic, non-conductive and heat-dissipating. It creates a protective layer for rapid heat dissipation, when applying the oil paint on an exterior wall.

FIG. 5 is another block diagram showing the application of the paint according to another preferred embodiment of the present invention. In this application, clean water, graphene, Shungite, and fermented boron nitride powder in 5 to 10 grams are added into paint coating to produce water paint having the heat-dissipating graphene. Preferably, the water paint is non-toxic, non-conductive and heat-dissipating. It creates a protective layer for rapid heat dissipation, when applying the water paint on an exterior wall.

When introducing elements of the present invention or the preferred embodiments thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A method of making paint having heat-dissipating graphene comprising steps of a first-time/second fermentation;
adding graphene mother bacteria to warm water and placing the graphene mother bacteria in a temperature-controlled room for multiple days to cultivate bacteria;
wherein the steps of the first-time/second fermentation include:
i) pouring the graphene mother bacteria, edible powders, and powdered sugars into a container, then adding the warm waters into the container so as to evenly stir the graphene mother bacteria, the edible powders, and the powdered sugars in the warm waters; placing the graphene baking powders in the temperature-controlled room and increasing a temperature in the temperature-controlled room for multiple days to ferment, thus graphene fermentation clump;
ii) crushing the graphene fermentation clump evenly to produce crushed graphene fermentation pieces, spreading the crushed graphene fermentation pieces on a platform and placing the crushed graphene fermentation pieces in the temperature-controlled room to be dried for multiple days, thus producing enzyme product;
iii) putting the enzyme product into a mixer so as to stir and refine the enzyme product into enzyme powders;
wherein after finishing a first-time fermentation, repeating the steps i) to iii) to complete the steps of the second-time fermentation;
wherein the second-time fermentation includes the steps of:
adding fermented graphene, fermented Shungite and fermented boron nitride into the warm water and placing the fermented graphene, the fermented Shungite, the fermented boron nitride, and the warm water in the temperature-controlled room for multiple days to cultivate the bacteria;
pouring the fermented graphene, the fermented Shungite, the fermented boron nitride, the edible powder and the sugar powder into the container, then adding the warm waters into the container so as to evenly stir the fermented graphene, the fermented Shungite, the fermented boron nitride, the edible powder and the sugar powder in the warm waters; then placing the graphene baking powders in the temperature-controlled room and increasing the temperature in the temperature-controlled room for multiple days to ferment, thus the graphene fermentation clump;
crushing the graphene fermentation clump evenly to produce the crushed graphene fermentation pieces, spreading the crushed graphene fermentation pieces on the platform and placing the crushed graphene fermentation pieces in the temperature-controlled room to dry for multiple days, thus producing the enzyme product; and
putting the enzyme product into the mixer so as to stir and refine the enzyme product into the enzyme powders, thus producing the paint having the heat-dissipating graphene.

2. The method as claimed in claim 1, wherein banana water, graphene, Shungite, and fermented boron nitride powder in 5 to 10 grams are added into oil paint coating to produce oil paint.

3. The method as claimed in claim 1, wherein clean water, graphene, Shungite, and fermented boron nitride powder in 5 to 10 grams are added into water paint coating to produce water paint

4. The method as claimed in claim 1, wherein the graphene mother bacteria is placed in the temperature-controlled room for two days to the cultivate bacteria.

5. The method as claimed in claim 1, wherein the graphene baking powders is placed in the temperature-controlled room for three days to ferment.

6. The method as claimed in claim 1, wherein the crushed graphene fermentation pieces are placed in the temperature-controlled room to dry for three to four days.

7. The method as claimed in claim 1, wherein the temperature-controlled room is at a temperature of 38° C. to 40° C.

8. The method as claimed in claim 1, wherein in the step i), a mixing proportion of the graphene mother bacteria, the edible powders, and the powdered sugars in the warm waters is the graphene mother bacteria in 150 grams, the edible powders in 3 kgs, the powdered sugars in 100 grams, and the warm water in 3000 cc.

9. The method as claimed in claim 1, wherein the platform is made of stainless steel.

10. The method as claimed in claim 1, wherein the container is a plastic blanket or a glass blanket.
